(19) United States
(12) Patent Application Publication
(10) Pub. No.: US 2004/0059387 A1
(43) Pub. Date: Mar. 25, 2004

(54) Low frequency acupuncture point electrically stimulating device

(76) Inventor: Tsung-I Yu, Taipei (TW)

Correspondence Address: BACON & THOMAS, PLLC, 625 SLATERS LANE, FOURTH FLOOR, ALEXANDRIA, VA, 22314

(21) Appl. No.: 10/403,126

(22) Filed: Apr. 1, 2003

Related U.S. Application Data
(63) Continuation-in-part of application No. 10/253,817, filed on Sep. 25, 2002.

(51) Int. Cl. A61N 1/00

(52) U.S. Cl. 607/2

(57) ABSTRACT

A low frequency acupuncture point electrically stimulating device comprises at least one pair of positive and negative electrodes, connected in series or in parallel so as to from as an electric stimulating piece; the electric stimulating piece being embedded into one of conductive films, copper foils and conductive rubber materials; and the positive and negative electrodes being connected to respective positive polarity joints and negative polarity joints through conductive paths; then the positive polarity joints and negative polarity joints being connected to respective plugs. The positive and negative electrodes are distributed according to the acupuncture points in predetermined positions on a bottom of a human foot.

### BACKGROUND OF THE INVENTION

(a) Field of the Invention

The present invention is a continuation in part (CIP) of U.S. patent series No. 10/253,817, filed at Sep. 25, 2002, which is incorporated into the present invention as a part of the present invention.

The present invention relates to massage device, and particularly to a low frequency acupuncture point electrically stimulating device which can be attached to brassieres, clothes, wrist strips, shoes, watches, eye masks, etc. A controller is used to control the positive and negative electrodes of an electric stimulating piece so as to actuate the massage function.

(b) Description of the Prior Art

The prior art low frequency electrically stimulating device for massage or medical treatment includes a controller, silicone positive and negative electrodes, or silicone soft glue (generally called as conductive glue), bowl shape silicone conductive clips, etc. The manufacturing cost is high and the applications thereof are confined in some areas with a bad conductive effect. Especially, the portions of body for being massaged or treated are coated with silicon soft glue. This will make the user feel uneasy and it is difficult to wash it. Besides, the silicone positive and negative electrodes and bowl shape silicone cups are too heavy to be worn on the body. Moreover, they are exposed out and occupy a larger space.

### SUMMARY OF THE INVENTION

The purpose of the present invention is for providing a low frequency acupuncture point electrically stimulating device which can be attached to brassieres, clothes, wrist strips, shoes, watches, eye masks, etc. A controller is used to control the positive and negative electrodes of an electric stimulating piece so as to actuate the massage function.

Another object of the present invention is to provide a low frequency acupuncture point electrically stimulating device which comprises at least one pair of positive and negative electrodes, connected in series or in parallel so as to from as an electric stimulating piece; said electric stimulating piece being embedded into one of conductive films, copper foils and conductive rubber materials; and the positive and negative electrodes being connected to respective positive polarity joints and negative polarity joints through conductive paths; then the positive polarity joints and negative polarity joints being connected to respective plugs. The positive and negative electrodes are distributed according to the acupuncture points in predetermined positions on a bottom of a human foot.

A yet object of the present invention is to provide a low frequency acupuncture point electrically stimulating device, wherein the conductive path is at a thin layer in the conductive film, and conductive films, copper foils and conductive rubber materials are soft or thin plate so that the present invention is light and occupies a small space.

A further object of the present invention is to provide low frequency acupuncture point electrically stimulating device, wherein the positive and negative electrodes can be controlled wired or wirelessly. The operation is easy.

A still object of the present invention is to provide a low frequency acupuncture point electrically stimulating device, wherein the electric stimulating piece can be attached to a brassiere at portions corresponding to the acupuncture points of the user.

A still object of the present invention is to provide a low frequency acupuncture point electrically stimulating device, wherein the electric stimulating piece can be attached to a shoe or shoe pad. The distribution of the electric stimulating piece is correspondent to the acupuncture points of foots.

A further object of the present invention is to provide a low frequency acupuncture point electrically stimulating device, wherein the electric stimulating piece is attached to a net-like clothes so as to stimulate the body of the user.

A further object of the present invention is to provide a low frequency acupuncture point electrically stimulating device, wherein the electric stimulating piece is combined to tension strips which are worn around the wrist.

A further object of the present invention is to provide a low frequency acupuncture point electrically stimulating device, wherein the electric stimulating piece is attached to a watch so as to stimulate the waist of the user.

A further object of the present invention is to provide a low frequency acupuncture point electrically stimulating device, wherein the electric stimulating piece is attached to eye mask for massaging eyes of the user.

### BRIEF DESCRIPTION OF THE DRAWINGS

FIG. 1 illustrates a partial section of an electric stimulating piece composed of at least one pair of positive and negative electrodes, connected in series or in parallel, embedded in a conductive film.

FIG. 2 is a cross-sectional view of the electric stimulating piece illustrated in FIG. 1.

FIG. 3 is a perspective view of the controller used with the electric stimulating piece.

FIG. 4 shows that the present invention is used to cups of woman brassiere.

FIG. 5 is a cross section view of FIG. 4.

FIG. 6 shows one embodiment of the present invention, wherein the electric stimulating piece is formed by positive and negative electrodes.

FIG. 7 shows another embodiment of the present invention, wherein the electric stimulating piece is formed by positive and negative electrodes.

FIGS. 8 to 11 show the conductive surfaces of the present invention are assembled in various ways.

FIG. 12 is a schematic view showing the electric stimulating piece of the present invention is embedded into an inner surface of a brassiere.

FIG. 13 shows that the electric stimulating piece of the present invention is integrally installed to the brassiere cups.

FIG. 14 show that in the present invention, a controller is used to control the positive and negative electrodes.

FIG. 15 is a schematic view showing the adjusting switch of the present invention which is at outer surfaces of brassiere cups.

FIGS. 16 to 21 show the embodiments of the present invention, where the electric stimulating pieces are arranged on the acupuncture points of human breast.

FIG. 22 is a schematic view showing that conductive films are used to form the electric stimulating pieces which is applied to the brassiere cups.

FIG. 23 is a schematic cross section view showing that in the present invention, copper film or conductive rubber is used to form electric stimulating piece which is used to brassiere cups.

FIG. 24 is a schematic view showing one embodiment about the wired control of the positive and negative electrodes of the present invention which are installed on a brassiere.

FIG. 25 is a schematic view showing another embodiment about the wired control of the positive and negative electrodes of the present invention which are installed on a brassiere.

FIG. 26 shows the embodiment that the electric stimulating piece is as sticky piece which is adhered to a brassiere.

FIG. 27 is a schematic cross section view of FIG. 26.

FIG. 28 is a schematic view of FIG. 26 showing the application of the present invention.

FIG. 29 is a schematic view showing that the present invention is applied to net-like clothes.

FIG. 30 is a schematic view showing that the present invention is applied to a bodysuit.

FIG. 31 is a schematic view showing that the present invention is used to man trousers.

FIG. 32 is a schematic view showing that the present invention is used to woman trousers.

FIG. 33 is a schematic view showing that the present invention is used to a conductive film of net-like trousers.

FIG. 34 is a schematic view showing that the present invention is used to another conductive film of net-like trousers.

FIG. 35 is a schematic view showing that a surface of the conductive film of the present invention has conductive tips.

FIG. 36 is a schematic view about the application of the tips of the present invention, wherein the tips serve to remove the hairs of human body.

FIG. 37 is a schematic view showing the present invention being applied to a tension strip.

FIG. 38 is a schematic view showing that the present invention is used to another kind of tension strip.

FIG. 39 is a schematic view showing that the present invention is used to a further kind of tension strip.

FIG. 40 is a schematic view showing that the present invention is used to a further kind of tension strip.

FIG. 41 is a schematic view showing the acupuncture points of human foot.

FIG. 42 shows the embodiment of the present invention where the present invention is applied to a shoe pad.

FIG. 43 is an embodiment of the present invention, wherein the present invention is applied to a shoe.

FIG. 44 shows one embodiment of the present invention, wherein the present invention is applied to a massage treading pad.

FIGS. 45 to 48 show embodiment of the present invention, wherein electric stimulating pieces are adhered to shoes.

FIG. 49 shows one embodiment of the present invention, wherein the present invention is used to a shoe.

FIG. 50 shows another embodiment of the present invention, wherein the present invention is used to a shoe.

FIG. 51 shows a further embodiment of the present invention, wherein the present invention is used to a shoe.

FIG. 52 shows one the present invention that the present invention is applied to a massage treading pad.

FIG. 53 is an embodiment showing that the present invention is used to a watch.

FIG. 54 is a lateral view of the present invention.

FIG. 55 shows one embodiment that the present invention is applied to a massage eye mask.

FIG. 56 is a schematic view of FIG. 55 which is view from another orientation.

### DETAILED DESCRIPTION OF THE PREFERRED EMBODIMENTS

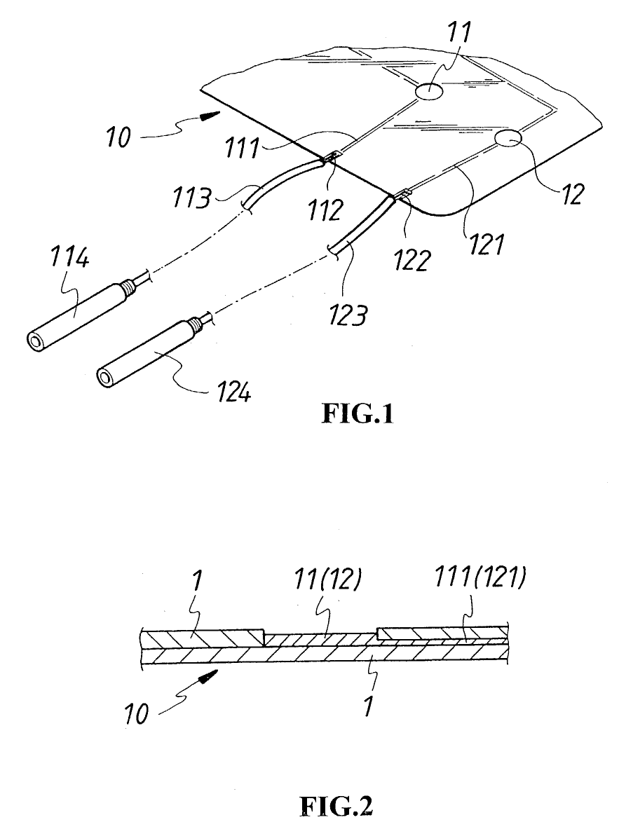

As shown in FIGS. 1 and 2, this present invention is related to a lower frequency electric stimulating acupuncture point massage device. The lower frequency electric stimulating acupuncture point massage device comprises at least one pair of positive and negative electrodes 11 and 12 is built into a conductive film 1, made of copper foils or conductive rubber materials, and connected in series or in parallel to form an electric stimulating piece 10. In more detail, the positive and negative electrodes 11 and 12 are respectively connected to electrode joints 112 and 122 through conductive paths 111 and 121. The electrode joints 112 and 122 are further respectively connected to conductive inserting pins 114 and 124 through wires 113 and 123. With a distance apart, the conductive paths 111 and 121 can be fixed within the conductive film 1 between two thin foils so that the positive and negative electrodes 11 and 12 are in a state of insulation.

The above-mentioned electric stimulating piece 10, composed of at least one pair of the positive and negative electrodes 11 and 12 embedded in a conductive film 1 (made of copper foils or conductive rubber materials), so that the selected position of human body aligns to the positive and negative electrodes 11 and 12.

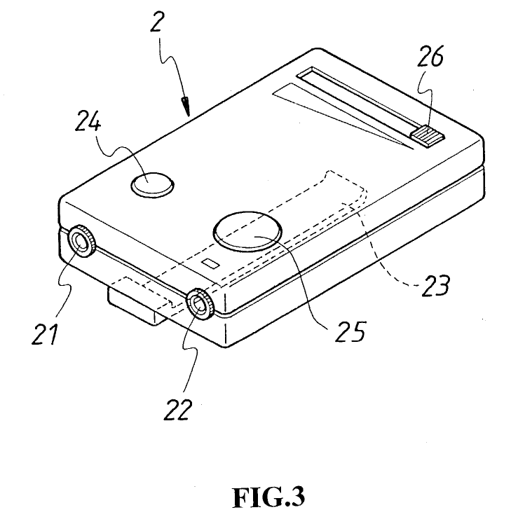

Meanwhile, low frequency electromagnetic waves are activated, at the pairs of positive and negative electrodes 11 and 12 by a wired or wireless controller 2 as shown in FIG. 3, to stimulate electrically the area about an acupuncture point. The electric stimulation provides an effect of massage that improves the blood circulation in the body portion the electric stimulating piece 10 is pressed on, eventually leading an effect of promoting one's health.

The functions of the above-mentioned controller 2 are at least switching on/off an output current and regulating the magnitude of the current. Control switches 24 and 25 are for starting and stopping the control and an adjusting switch 26 is for regulating the output current; the adjusting switch 26 controls a stepless adjustment by a knob or a push-pull piece. The controller 2 contains batteries and the conductive inserting pins 21 and 22 thereof are connected to the conductive inserting pins 114 and 124 of the electric stimulating piece 10. Further, a clip 23 can be installed at a chosen area on the controller 2 for wearing on clothes, if desired.

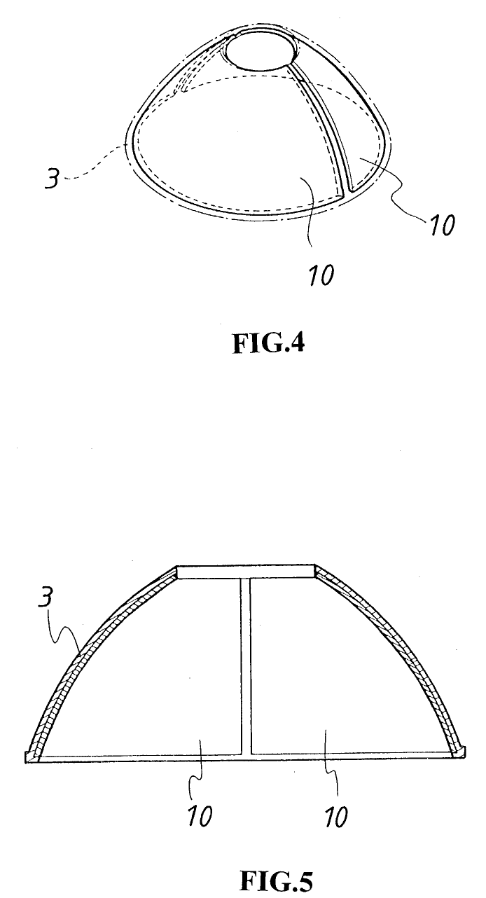

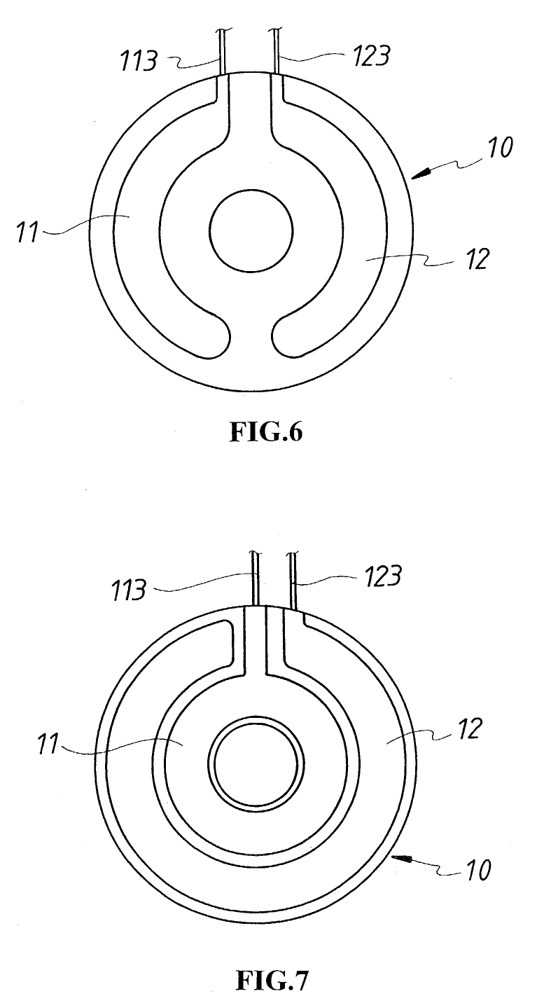

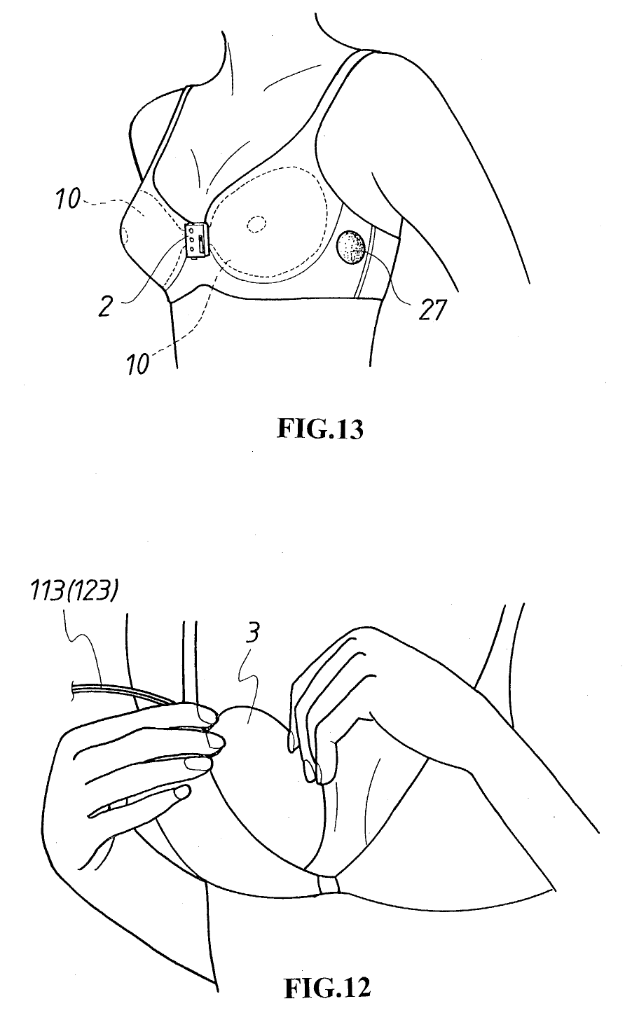

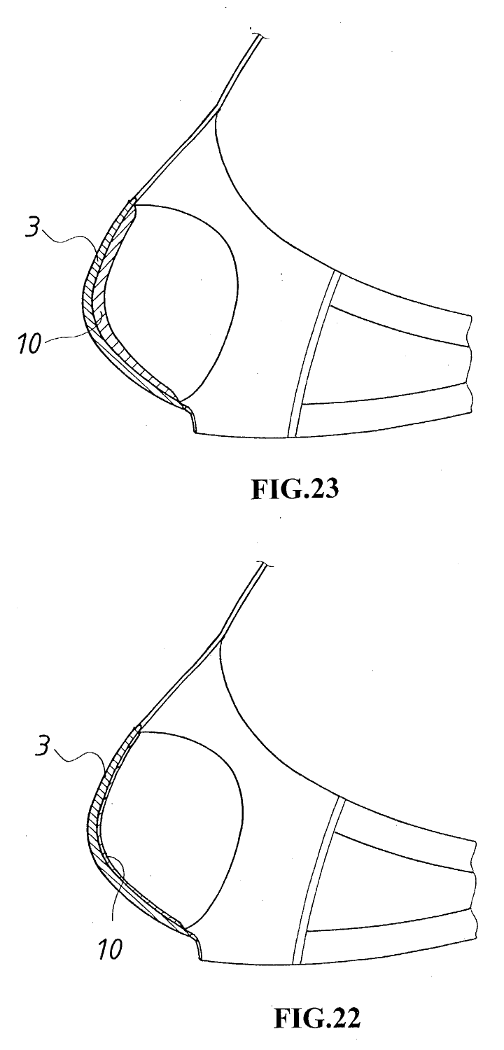

Referring to FIGS. 4 and 5, the electrically stimulating piece 10 is used in the brassiere cup 3 (referring to FIGS. 12 to 28) so that the positive electrode 11 and negative electrode 12 are positioned corresponding to the breast acupuncture points. Thereby, in conduction, the breast acupuncture points can be electrically stimulating so as to beautify the breast of the user. In the present invention, the distribution of the positive electrode 11 and negative electrode 12 of the electrically stimulating piece 10 in the cup 3 is not confined in the present invention. Referring to FIGS. 6 and 7, if the electrically stimulating piece 10 with different the positive electrode 11 and negative electrode 12 is positioned at the illustrated position, swelling of the breast can be removed.

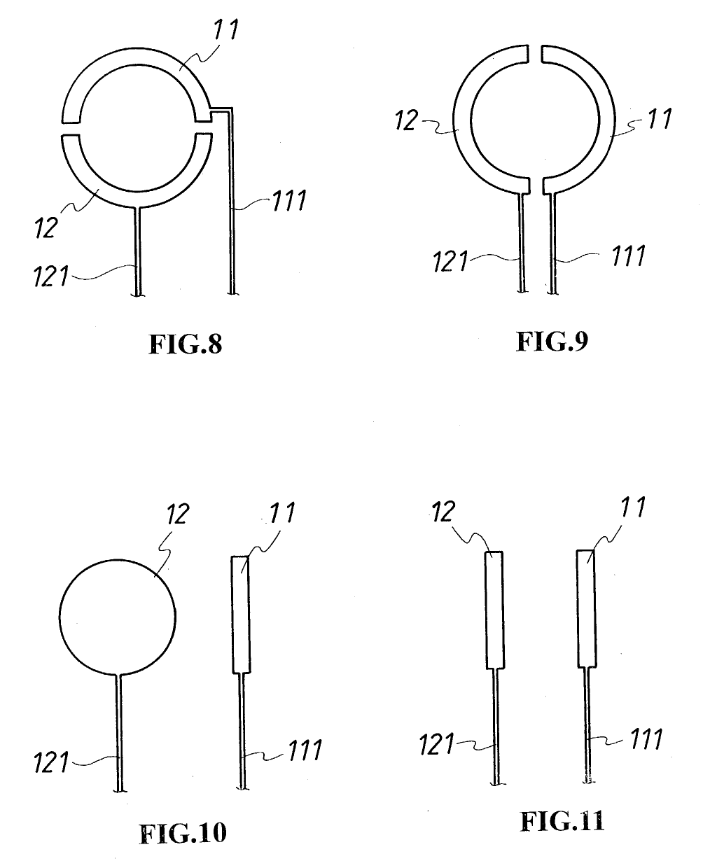

Referring to FIGS. 8 to 11, the conductive film 1 is suitable to be mass-produced by using printed circuit board. The surface of the circuit is installed with at least one set of positive and negative electrodes 11 and 12 and conductive paths 111, 121. The positive and negative electrodes 11 and 12 may have cambered shapes, round shapes, or strip shapes.

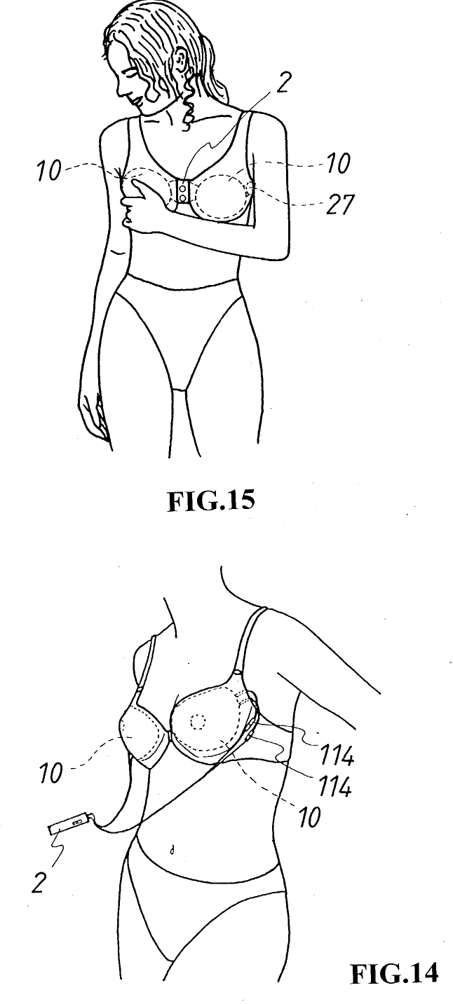

The electrically stimulating piece 10 in the cup 3 can be adhered to an inner surface of one cup 3 (referring to FIG. 12). A wired or wireless controller 2 can be used to control any set of positive electrode 11 and negative electrode 12 (referring to FIG. 14). By low frequency to electric-stimulating breast acupuncture point portions of a human body, the circulation of blood can be improved and the breast of the user can be enlarged. Moreover, an adjusting switch 27 for controlling current can be installed at an outer side of the brassiere so that it can be operated conveniently (referring to FIG. 15).

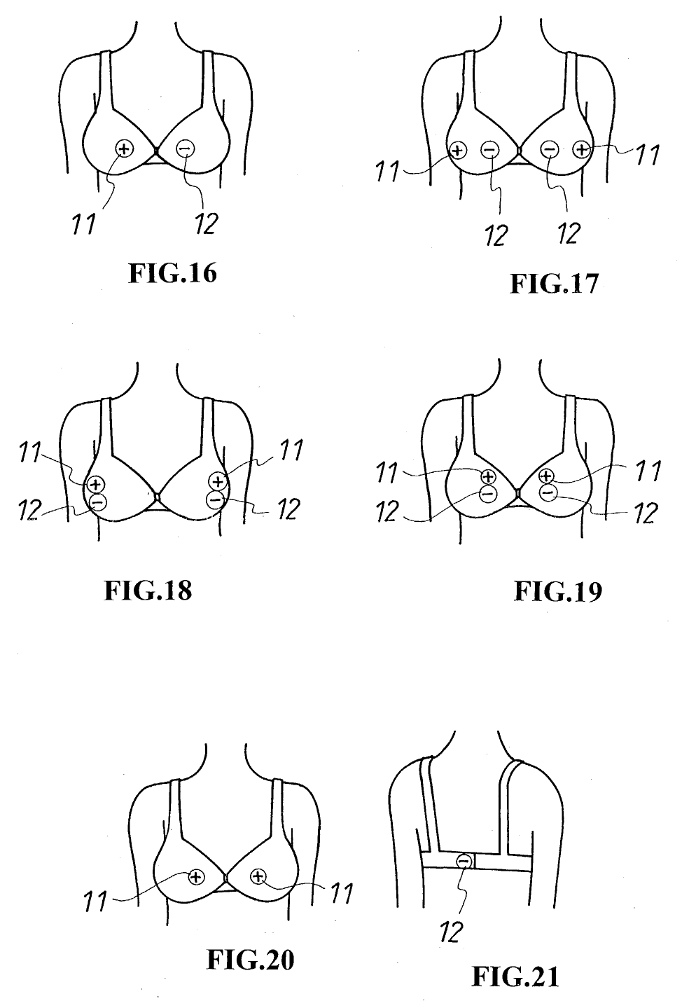

Referring to FIGS. 16 and 21, another embodiment of the present invention is illustrated. In that, the electric stimulating piece 10 can be distributed at the selected acupuncture points.

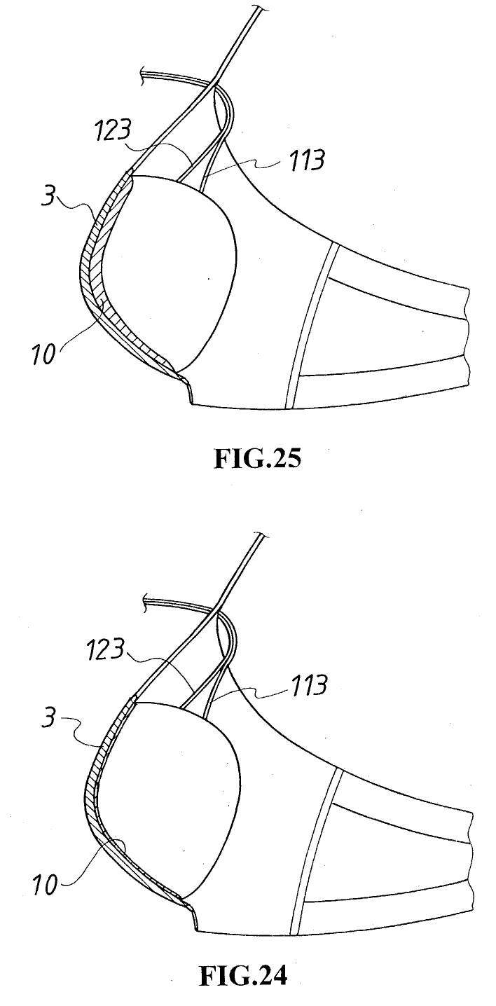

In the present invention, the electric stimulating piece 10 used in the cup 3 is not limited to the conductive film as illustrated in FIG. 3. An inner placed electric stimulating piece 10 made of copper film plate or conductive rubber can be placed at the inner surface of the cup 3. Wire (referring to FIGS. 24, 25) or wireless way can be used to control the electric stimulating piece 10.

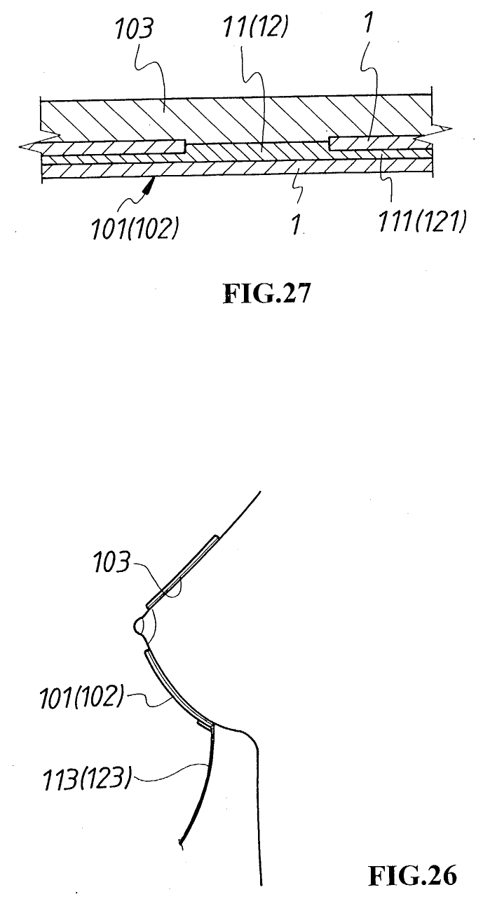

Referring to FIGS. 26 to 28, the electrically stimulating piece 10 used in the woman breast can be made as separated positive electrode piece 101 and negative electrode piece 102. Each piece is coated with a conductive silicon rubber 103 for being adhered to the breast of the woman so that low frequency massage can be performed.

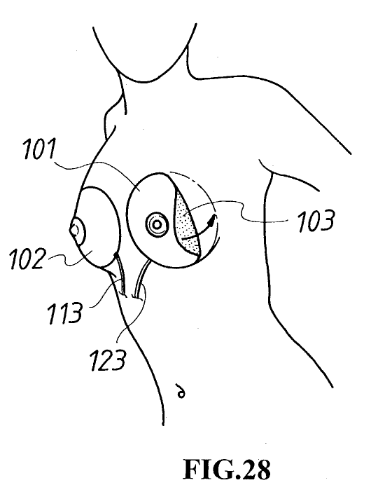

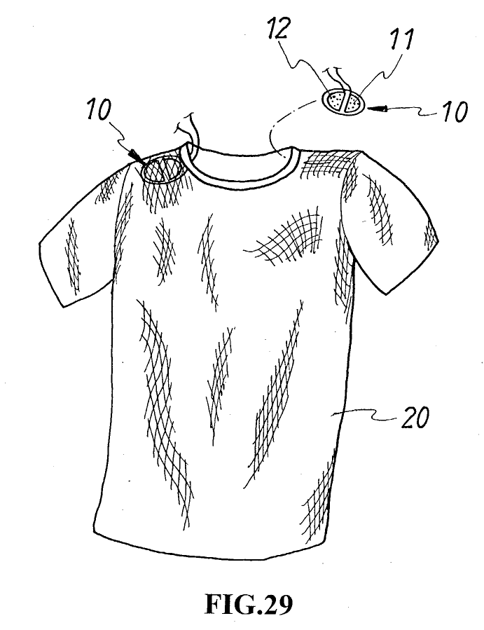

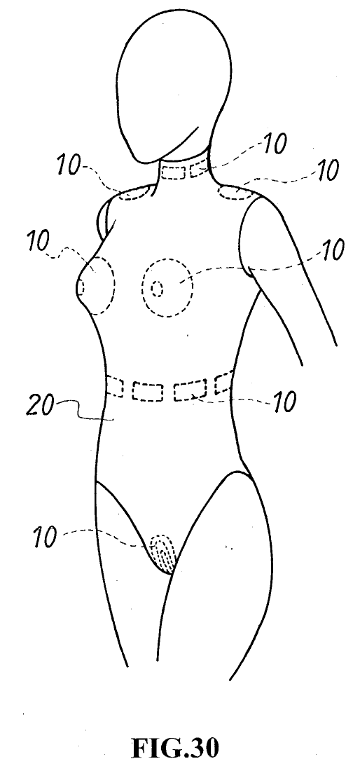

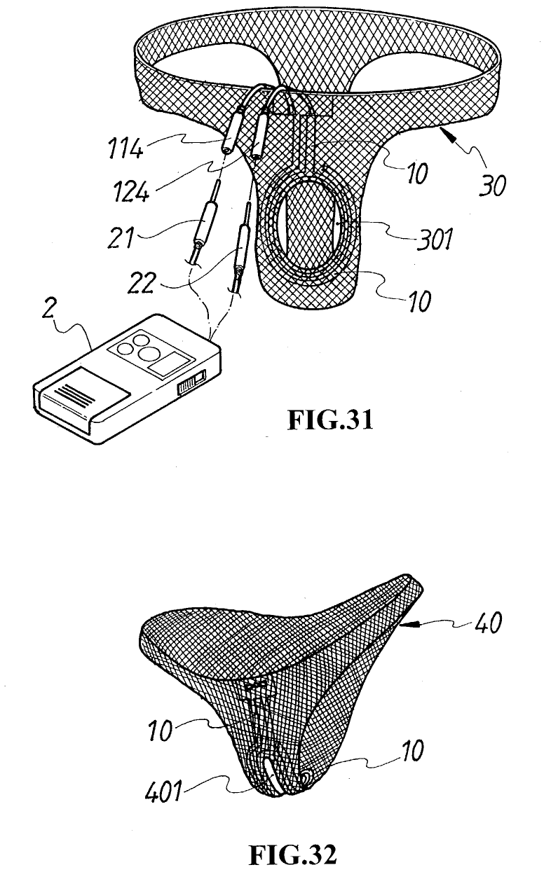

Referring to FIG. 28, the electric stimulating piece 10 of the present invention can be applied to net-like clothes 20 so that the electric stimulating piece 10 is adhered to the net-like clothes 20 of shoulders, breast, back or wrist. Thereby, in wearing, the electric stimulating piece 10 vibrates so as to stimulate acupuncture points. Referring to FIG. 30, the net-like clothes 20 can be formed to enclose the whole body, while electric stimulating pieces 10 are placed on the neck, shoulders, breasts, wrist, etc.

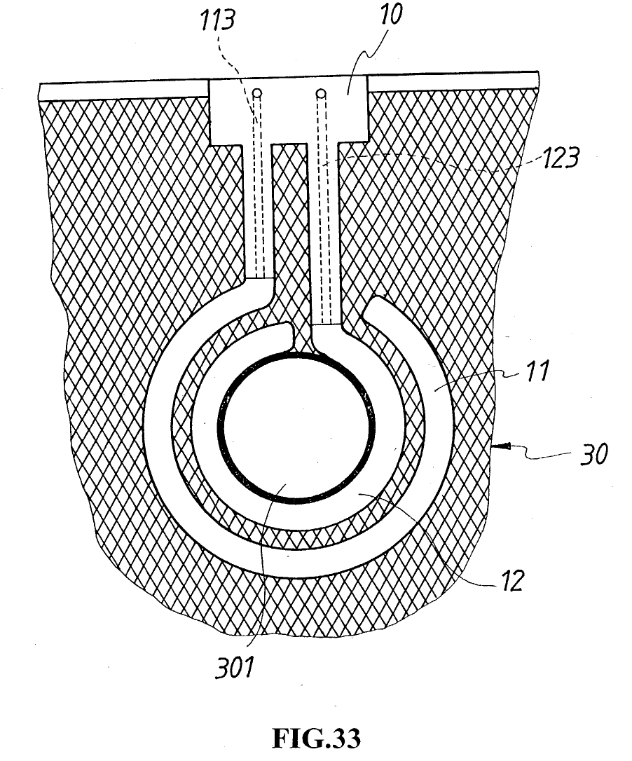

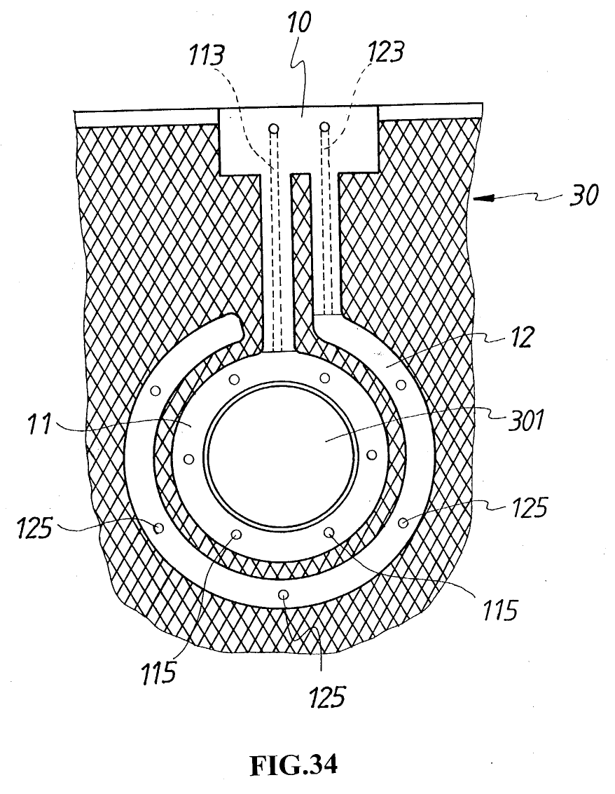

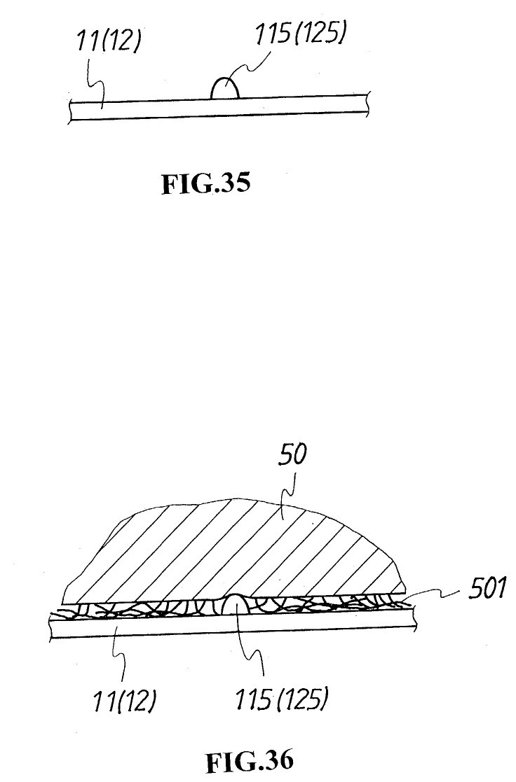

As shown in FIGS. 32, and 32, the electric stimulating pieces 10 are adhered to the flexible net-like trousers 30 or 40. The genital portions of the net-like trousers 30 or 40 are formed with via holes 301, 401. The inner peripheries of the via holes 30, 40 are installed with circle-like electric stimulating pieces 10 (referring to FIGS. 33 and 34). The positive and negative electrodes 11 and 12 of the electric stimulating pieces 10 are formed with a plurality of tips 115, 125. With reference to FIG. 35, each tip 115 or 125 may pass through hairs 50 of genitals or hairs. 501 of body so as to contact the body (referring to FIG. 36).

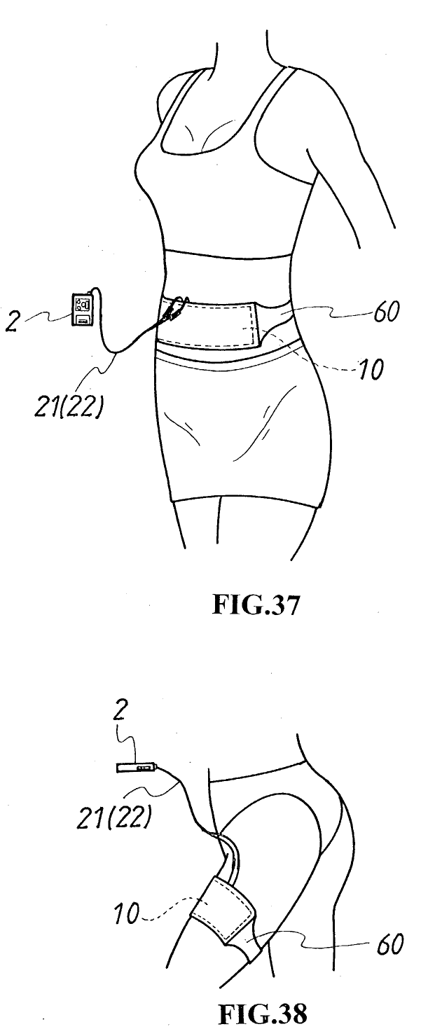

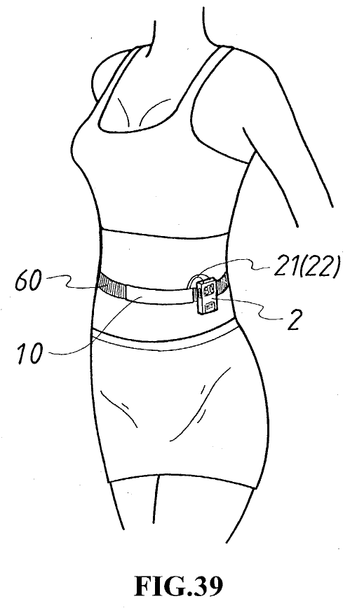

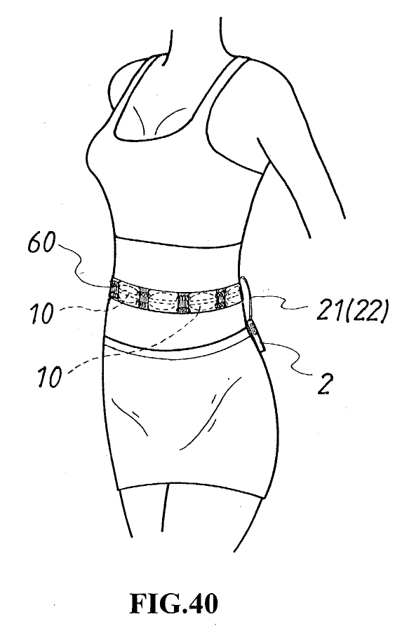

Referring to FIGS. 37, 40, the electric stimulating pieces 10 can be adhered to inner surfaces of various tension strips 60 for enclosing the hands, or legs, or wrists, etc. so as to stimulate the body.

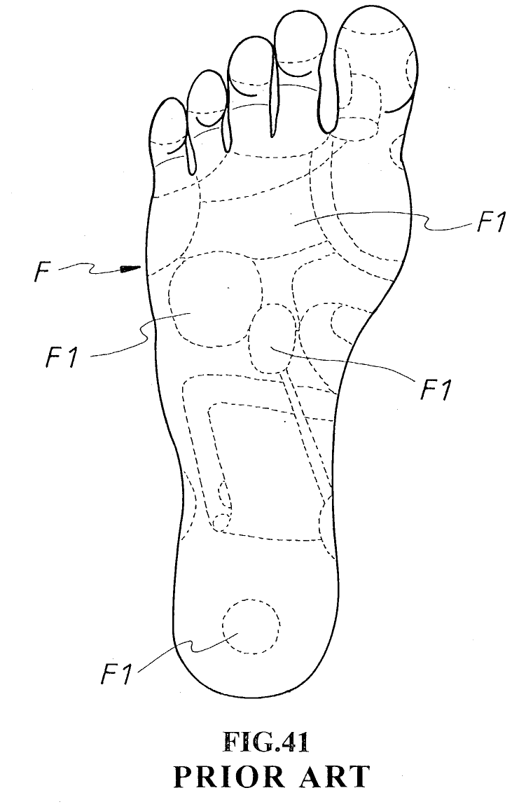

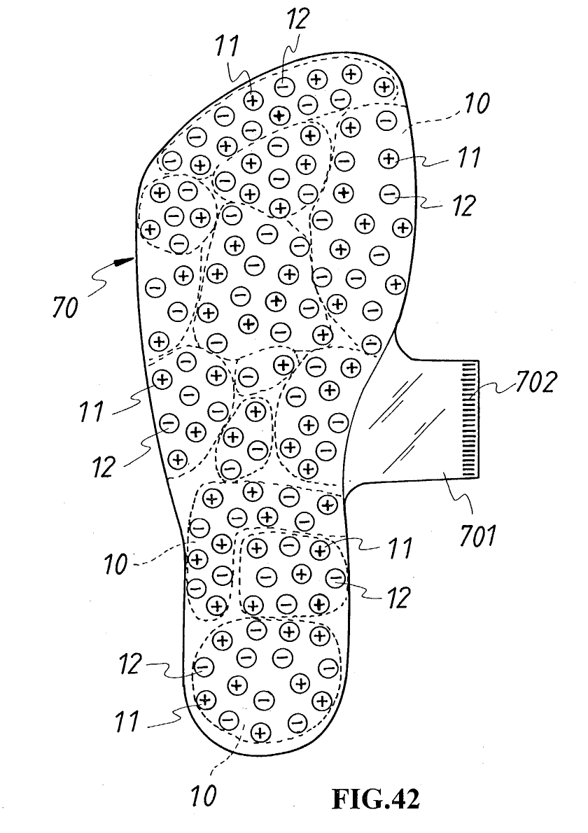

Referring to FIGS. 41 and 42, the electric stimulating piece 10 is adhered to a shoe pad 70 (in FIG. 42) based on the reflecting sections F1 illustrated in FIG. 41. One or more pairs of positive and negative electrodes 11, 12, corresponding to the acupuncture points in various reflex areas on the bottom of a foot, are embedded within the electric stimulating piece 10, connected either in parallel or in series. The wires connected to those electrodes are converged to a lateral piece 701 to form a BUS wire portion 702, which portion is for connecting to a wired controller 2.

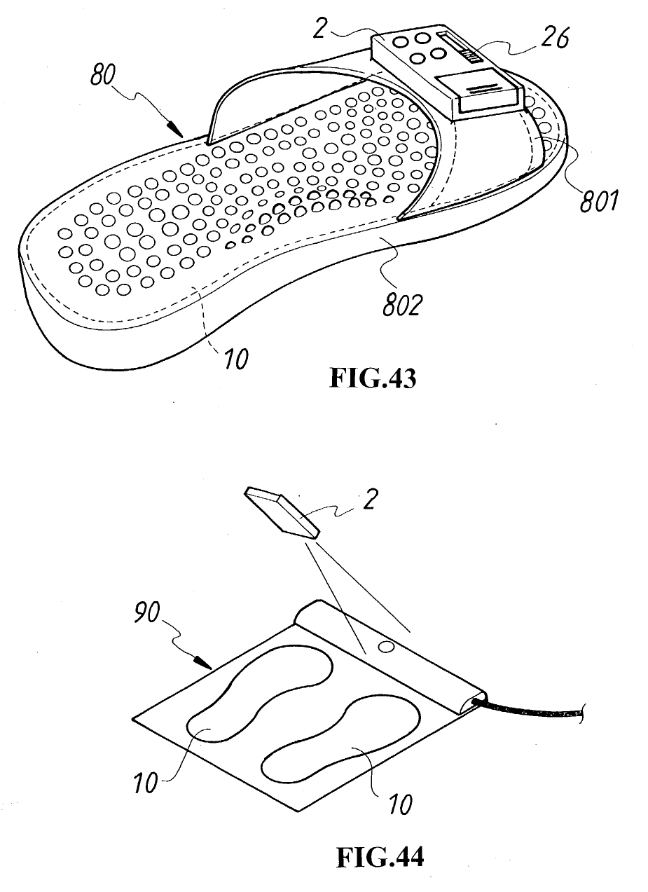

The electric stimulating piece 10 is adhere to a shoe 80 (referring to FIG. 43) or as a massage pad 90 (referring to FIG. 44). A wired or wireless controller 2 (which can be fixed to the wearing surface 801 of a shoe) can be selected for controlling the positive and negative electrodes. Thereby, the acupuncture points in the foot can be stimulated by low frequency.

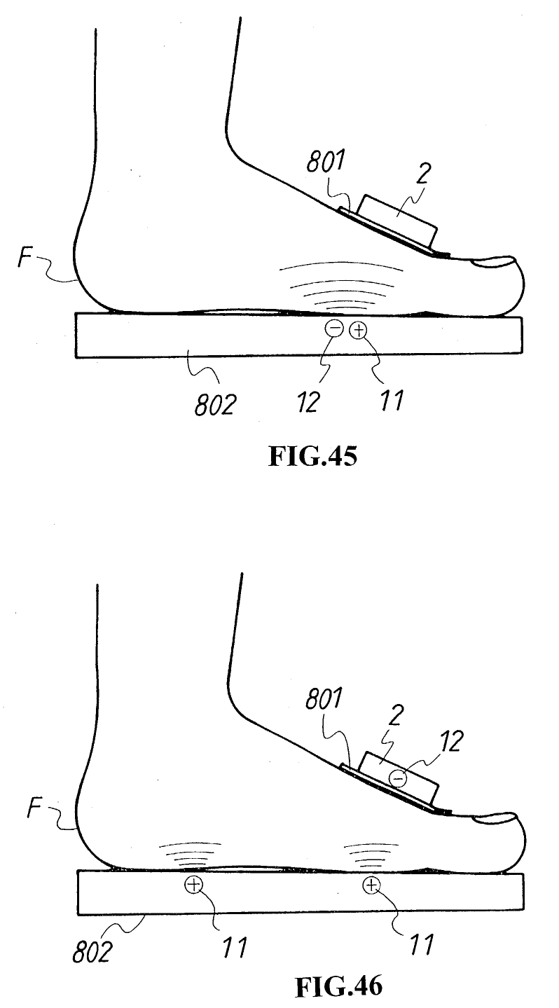

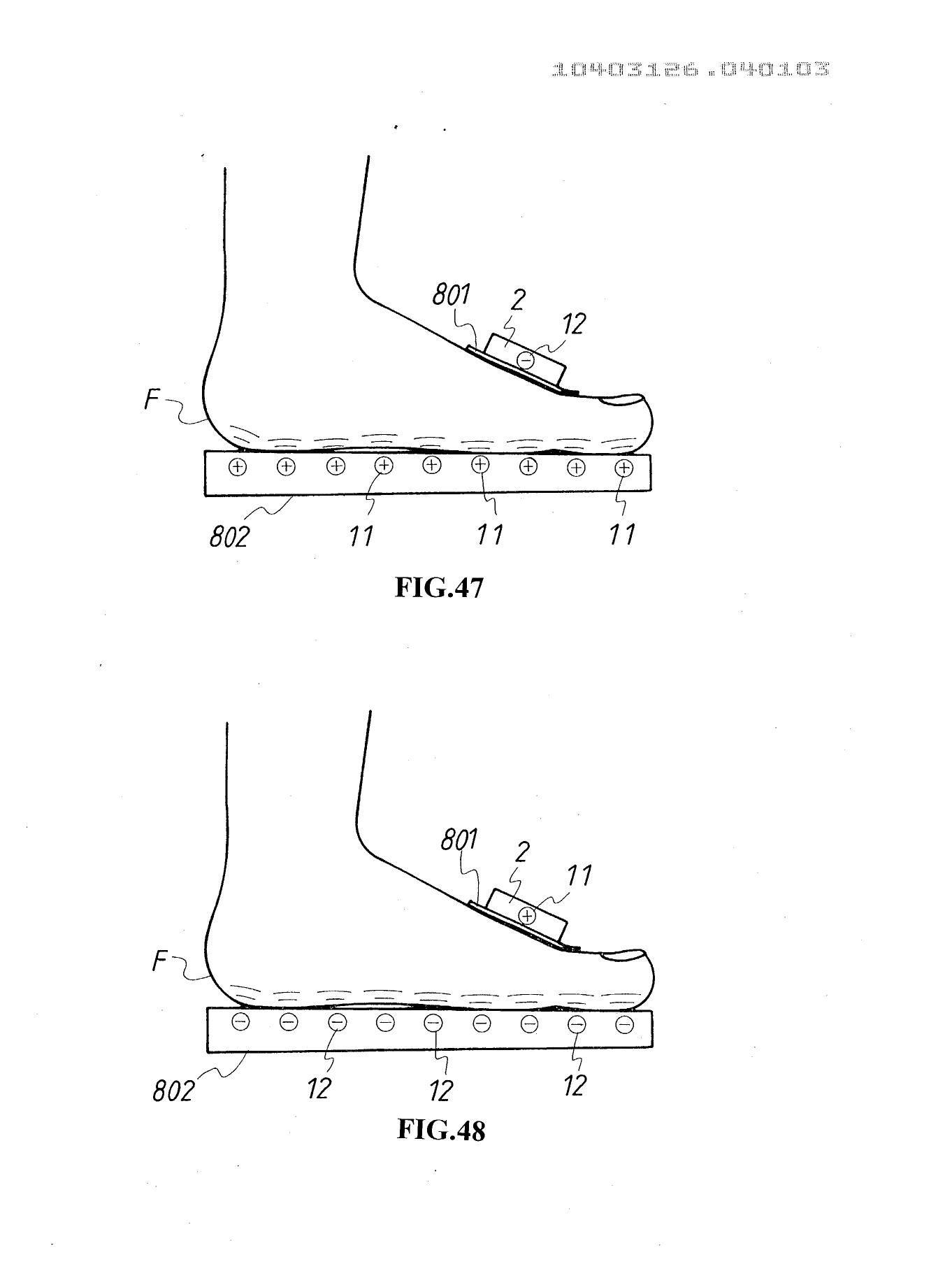

When the present invention is applied to a shoe 80, the upper and lower wearing surfaces 801 and 802 are assembled with a plurality of positive or negative electrodes, such as those illustrated in FIGS. 45 to 48.

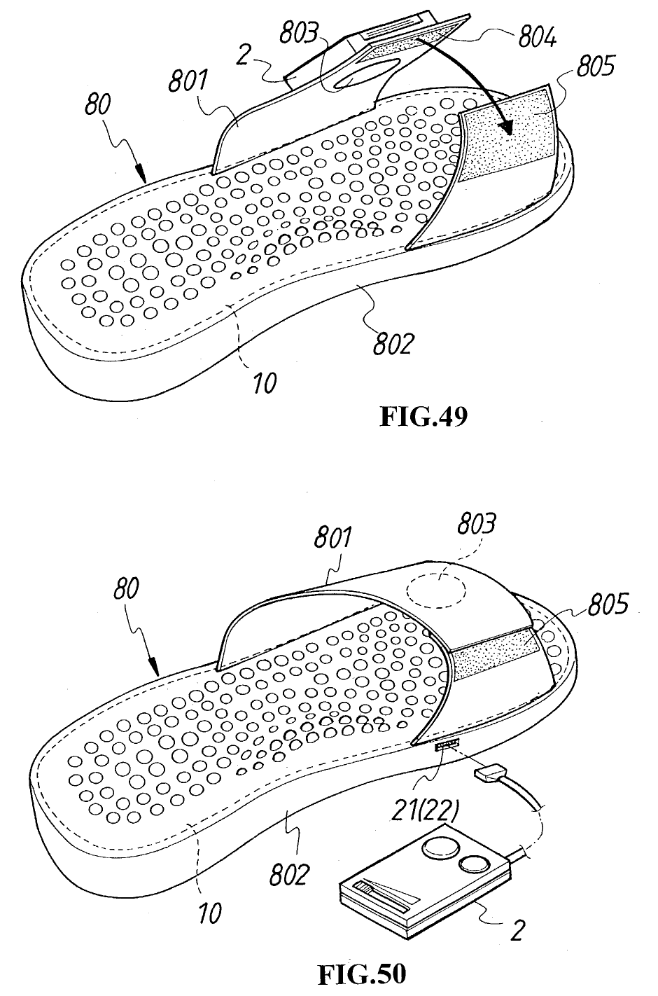

With reference to FIG. 49, when the present invention is applied to a shoe 80, the inner wall of the upper wearing surface 801 is assembled with a positive or negative electrode 803 which is matched to the positive or negative electrode 11 or 12 of the electric stimulating piece 10 so as to have electrically massage effect. In one the present invention, the upper wearing surface 801 of the shoe 80 is formed by two sticky pieces 804, 805.

Referring to FIG. 50, when the present invention is applied to a shoe 80, the electrode 21 or 22 of an external controller 2 is installed at the lower wearing surface 802 so that the controller 2 controls the massage in low frequency.

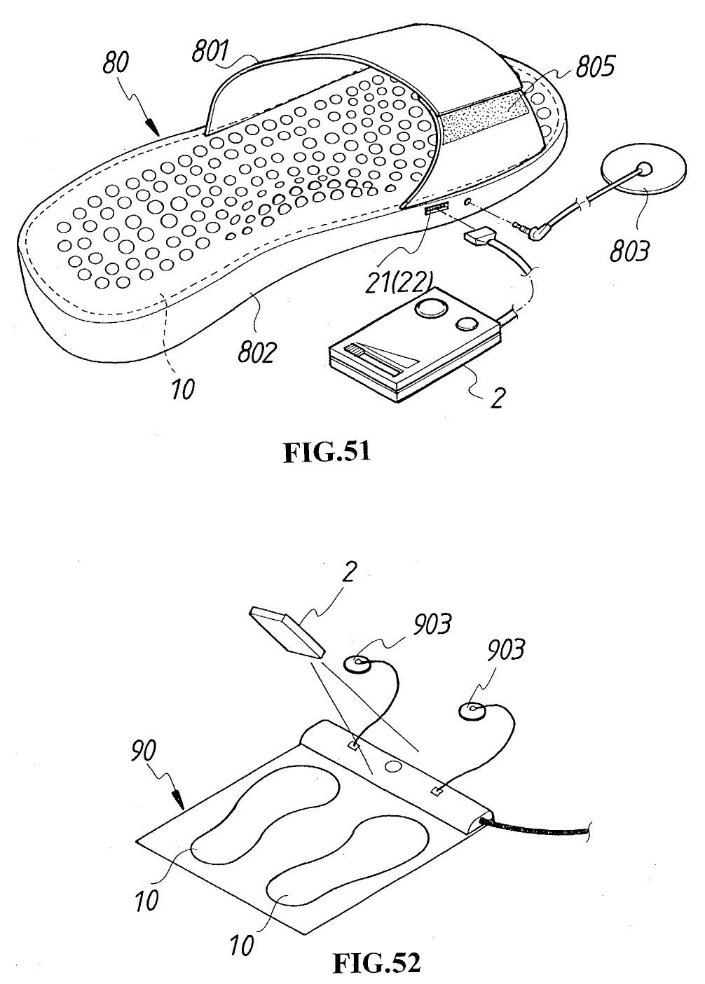

As shown in FIG. 51, an external positive or negative electrode 803 is assembled to the shoe 80 so that the positive or negative electrodes 803 are adhered on the upper end of the foot so that the external controller 2 is installed to the lower wearing surface 802. Thereby, the external controller can control the lower frequency massage operation

With reference to FIG. 52, the present invention is applied to a massage treading pad 90, two extended polarity pads 903 are installed on the massage treading pad 90. The two pads 903 can be adhered on foots for massage by low frequency electrically wave.

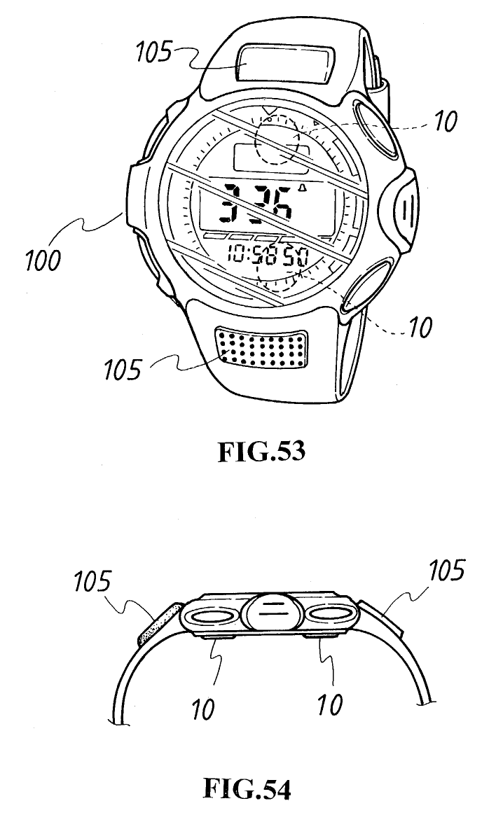

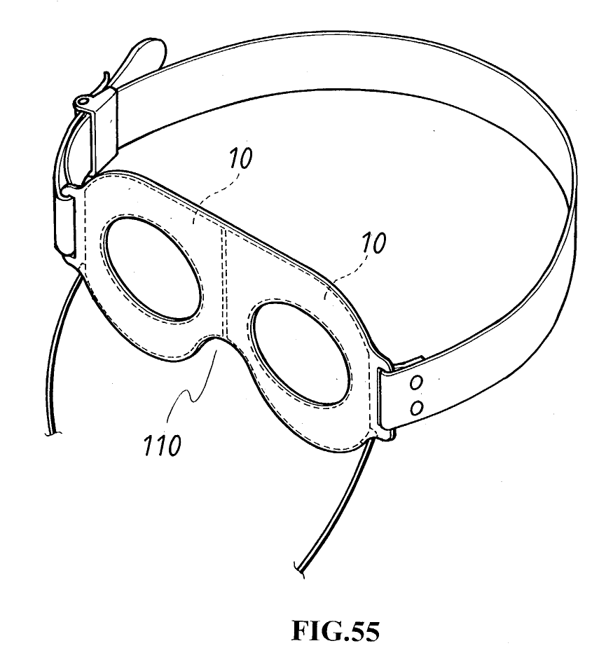

With reference to FIGS. 53 and 54, the electric stimulating pieces 10 are attached to a watch 100 so as to touch the waists. Each electric stimulating piece 10 has at least one positive and negative electrodes 11 and 12. A control switch 105 is installed on the watch 105 for actuating the low frequency massage.

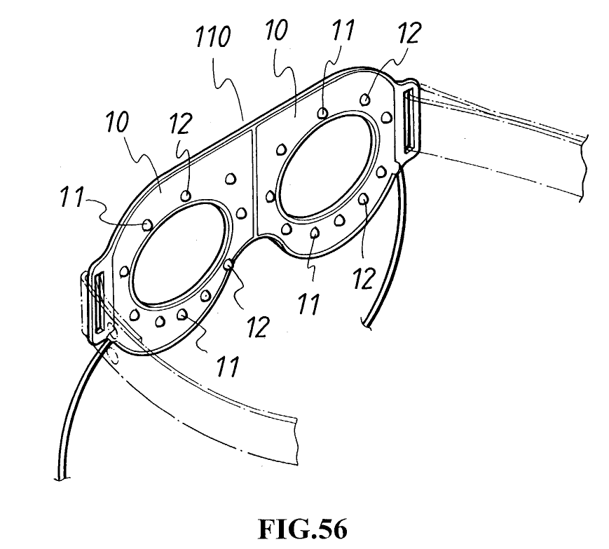

As shown in FIGS. 55 and 56, at least one set of electric stimulating piece 10 having positive and negative electrodes 11 and 12 is attached to inner surfaces of an eye mask 110. The positive and negative electrodes 11 and 12 is wired or wirelessly controlled by a controller. Thereby, the electric stimulating pieces can massage human eyes.

The various application configurations of a low frequency foot-electric stimulating assistor illustrated above are only embodiments of the present invention, which should not restrict the technical field of the present invention. Other equivalent applications made by obvious changes based on the same techniques should be included in the technical field of the present invention.

What is claimed is:

1. A low frequency acupuncture point electrically stimulating device comprising:
at least one pair of positive and negative electrodes, connected in series or in parallel so as to from as an electric stimulating piece; said electric stimulating piece being embedded into one of conductive films, copper foils and conductive rubber materials; and the positive and negative electrodes being connected to respective positive polarity joints and negative polarity joints through conductive paths;
then the positive polarity joints and negative polarity joints being connected to respective plugs;
wherein said positive and negative electrodes are distributed according to the acupuncture points in predetermined positions on a bottom of a human foot.

2. The low frequency acupuncture point electrically stimulating device as claimed in claim 1 wherein the conductive path is at a thin layer in the conductive film.

3. The low frequency acupuncture point electrically stimulating device as claimed in claim 1, wherein conductive films, copper foils and conductive rubber materials are soft or thin plate.

4. The low frequency acupuncture point electrically stimulating device as claimed in claim 3, wherein conductive films, copper foils and conductive rubber materials are printed circuit with different positive and negative electrodes and conductive paths.

5. The low frequency acupuncture point electrically stimulating device as claimed in claim 1, wherein currents of the positive and negative electrodes are wired or wireless controlled.

6. The low frequency acupuncture point electrically stimulating device as claimed in claim 1, wherein the at least electric stimulating piece is attached to the cup or interior of a brassiere.

7. The low frequency acupuncture point electrically stimulating device as claimed in claim 1, wherein the positive and negative electrodes of each electric stimulating piece has one of shapes containing cambered shapes, round shapes, and long strips.

8. The low frequency acupuncture point electrically stimulating device as claimed in claim 6, wherein an outer surface of the brassiere is installed with an adjusting switch for controlling current strength.

9. The low frequency acupuncture point electrically stimulating device as claimed in claim 1, wherein the positive and negative electrodes are sticky pieces

10. low frequency acupuncture point electrically stimulating device as claimed in claim 1, wherein the electric stimulating piece is adhered to an inner surface of a net-like clothes.

11. The low frequency acupuncture point electrically stimulating device as claimed in claim 10, wherein the net-like clothes is a bodysuit.

12. The low frequency acupuncture point electrically stimulating device as claimed in claim 1, wherein the electric stimulating piece is adhered to a pair of net-like trousers.

13. The low frequency acupuncture point electrically stimulating device as claimed in claim 12, wherein the net-like trouser has via holes, and peripheries of the via holes are installed with electric stimulating pieces.

14. The low frequency acupuncture point electrically stimulating device as claimed in claim 13, wherein the positive and negative electrodes of the electric stimulating piece have a plurality of conductive tips.

15. The low frequency acupuncture point electrically stimulating device as claimed in claim 1, wherein the electric stimulating piece is adhered to an inner surface of a strip.

16. The low frequency acupuncture point electrically stimulating device as claimed in claim 1, wherein the electric stimulating piece is adhered to a shoe pad.

17. The low frequency acupuncture point electrically stimulating device as claimed in claim 16 wherein electric stimulating piece is matched to the acupuncture points of feet.

18. The low frequency acupuncture point electrically stimulating device as claimed in claim 16, wherein the positive and negative electrodes of the electric stimulating pieces are at one side so as to form as a bank.

19. The low frequency acupuncture point electrically stimulating device as claimed in claim 1, wherein the electric stimulating pieces are adhered to a shoe.

20. The low frequency acupuncture point electrically stimulating device as claimed in claim 1, wherein the electric stimulating pieces are adhered to a massage treading pad.

21. The low frequency acupuncture point electrically stimulating device as claimed in claim 19, wherein the upper and lower wearing surfaces are installed with a plurality of positive and negative electrodes.

22. The low frequency acupuncture point electrically stimulating device as claimed in claim 19, wherein an inner surface of the upper wearing surface of the shoe is installed with the positive or negative electrodes.

23. The low frequency acupuncture point electrically stimulating device as claimed in claim 19, wherein the upper wearing surface is formed by two sticky pieces.

24. The low frequency acupuncture point electrically stimulating device as claimed in claim 19, wherein the lower wearing surface is installed with polarity joints for being connected to an external controller.

25. The low frequency acupuncture point electrically stimulating device as claimed in claim 19, wherein the upper wearing surface of the shoe has an external electrodes and the lower wearing surface is installed with an external controller.

26. The low frequency acupuncture point electrically stimulating device as claimed in claim 20, wherein the massage treading pad is extended with two polarity sticky pieces.

27. The low frequency acupuncture point electrically stimulating device as claimed in claim 1, wherein the electric stimulating piece is adhered to a watch.

28. The low frequency acupuncture point electrically stimulating device as claimed in claim 27, wherein the watch has a control switch.

29. The low frequency acupuncture point electrically stimulating device as claimed in claim 1, wherein the electric stimulating pieces are adhered to an inner surface of a massage eye mask.
